(19) United States
(12) Patent Application Publication
(10) Pub. No.: US 2017/0071894 A1
(43) Pub. Date: Mar. 16, 2017

(54) CHEMICAL STERILIZATION OF MALE RUMINANTS

(71) Applicant: Cowboy Animal Health, LLC, McKinney, TX (US)

(72) Inventor: Min Wang, Columbia, MO (US)

(21) Appl. No.: 14/855,041

(22) Filed: Sep. 15, 2015

(51) Int. Cl. A61K 31/315; A61K 9/00; A61K 9/08

(52) U.S. Cl. CPC A61K 31/315; A61K 9/08; A61K 9/0034

(57) ABSTRACT

A method of performing a non-surgical sterilization of a male animal is disclosed. The method of sterilization includes injecting a chemical sterilant into a testicle of the animal. The chemical sterilant contains an aqueous solution of a pharmaceutically acceptable zinc salt or mixtures thereof having a pH in the range of about 6 to 8. A chemical sterilant that is a solution of a pharmaceutically acceptable zinc salt or mixtures thereof having a pH in the range of about 6 to 8 is also disclosed.

### BACKGROUND

The technique of castration in domestic animals has been utilized for centuries. Primarily, castration is performed to reduce aggressiveness, to facilitate handling, to prevent unwanted breeding, and to improve the quality of the animal's meat. In the food industry, millions of male calves throughout the world are castrated every year. It is well-known that bulls produce lower quality grade, less consistent, less marbled, and less tender meat. As such, bulls command lower prices at market when compared with steers and are less desirable.

The testicles in male animals produce sperm and hormones, which affect growth and behavior. Castration can be accomplished by removing the testicles surgically, damaging them irreparably, or causing them to atrophy by stricture of the blood supply. The most common method of castration is surgical castration where an incision is made in the skin surrounding the testicles, then the blood supply is sewn shut, next the blood supply is severed, then the testicle is physically removed from the animal's body through that incision. Testicles have a significant blood supply and care must be taken to ensure there is not excessive bleeding before or after surgery.

In addition to the risk of hemorrhage, there are other significant risks to surgical castration. For instance, during the surgery, which is usually performed outside or in a barn, an open wound is created, which can become infected or otherwise fail to heal properly. Another problem with surgical castration is the pain that the calf experiences from the incision itself and from the physical removal of the two testicles. Normally, the animals are not sedated, nor is any pain relief medication administered. Castration also causes decreased weight gain of the animals and there is an increased risk of respiratory illness. Also, castration presents a possible safety hazard to the surgeon as sharp surgical knives are used during the procedure.

Thus there exists a need for a safer method to castrate a male animal The present application solves this problem by providing an effective method of chemical castration.

### SUMMARY

This application is directed to a method of performing a non-surgical sterilization of a male animal. The method of sterilization includes injecting a chemical sterilant into a testicle of the animal. The chemical sterilant contains an aqueous solution of a pharmaceutically acceptable zinc salt having a pH in the range of about 6 to 8. In addition, this application is directed to a chemical sterilant that is a solution of a pharmaceutically acceptable zinc salt having a pH in the range of about 6 to 8.

### BRIEF DESCRIPTION OF THE FIGURES

FIG. 1. Effect of castration, castration method, and injectable castration chemical sterilant dose on serum testosterone concentrations (least squares means).

FIG. 2. Effect of castration, castration method, and injectable castration chemical sterilant dose on testis and scrotum thickness (least squares means).

FIG. 3. Photograph of a testicle of an intact bovine at approximately 8 months of age (400×).

FIG. 4. Photograph of a testicle of a Group 1 bovine at approximately 8 months of age (400×).

FIG. 5. Photograph of a testicle of Group 2 bovine at approximately 8 months of age (400×).

FIG. 6. Photograph of a testicle of Group 3 bovine at approximately 8 months of age (400×).

### DETAILED DESCRIPTION

The present invention contemplates a method of performing a non-surgical sterilization of a male animal that entails injecting an aqueous chemical sterilant into a testicle of the animal. This method alleviates the problems of prior art sterilization methods in that it is very effective, easy to perform, and quick and painless for the animal. In addition, as there is no surgery involved, there are no open wounds to become infected or hemorrhage. Moreover, male animals sterilized by this chemical sterilant method suffer no ill effects as to total bodyweight or average daily weight gain as compared to those sterilized by surgical castration methods.

In this method, the chemical sterilant is an aqueous solution of a pharmaceutically acceptable zinc salt or mixtures thereof having a pH in the range of about 6 to 8. The zinc salt or mixtures thereof can be any pharmaceutically acceptable zinc salt, including zinc acetate, zinc propionate, zinc butyrate, zinc formate, zinc gluconate, zinc glycolate, zinc lactate, and mixtures thereof. In one embodiment, the zinc salt comprise zinc acetate.

Besides the zinc salt, the chemical sterilant in this method can further include an additive or mixtures thereof. The additive can be a pharmaceutically acceptable preservative; buffer; dye; colorant; numbing agent; solvent; detergent; antibiotic; antioxidant; viscolyzing agent; polycation such as polymyxins and their derivatives; protamine; polymer of basic amino acids; insect cecropin; reptilian magainin; cationic leukocyte peptide such as defensins, bactenecins, and bactericidal/permeability-increasing protein; aminoglycoside; chelator such as EDTA, nitrilotriacetic acid, and sodium hexametaphosphate; or any other pharmaceutically acceptable additive that is known in the art. In another embodiment, the additive can be an amino acid such as alanine, valine, isoleucine, proline, glycine, serine, threonine, asparagine, glutamine, lysine, arginine, histidine, and mixtures thereof. In yet another embodiment, the additive is histidine. In still another embodiment, the additive is dimethyl sulfoxide. In this regard, the chemical sterilant can be a mixture of zinc acetate, histidine, and dimethyl sulfoxide, with a pH in the range of about 6 to 8.

The zinc salt or mixtures thereof is present in the chemical sterilant at a concentration in the range of about 70 to 100 mg/ml. The histidine is present at a concentration in the range of about 0.1 to about 0.3 M. The dimethyl sulfoxide is present at a range of about 0.1-70% by volume.

Although animals may be sterilized with the present chemical sterilant at any age, most often sterilization is done before the animal is sexually mature. In one embodiment, the male animal is sterilized with the present chemical sterilant at an age between 3-24 months.

The method and chemical sterilant described herein are applicable to most types of male animals. In one embodiment, the method and chemical sterilant can be applied to a male bull calf.

The chemical sterilant of the present application includes an aqueous solution of a pharmaceutically acceptable zinc salt or mixtures thereof having a pH in the range of about 6 to 8. The zinc salt or mixtures thereof can be any pharmaceutically acceptable zinc salt, including zinc acetate, zinc propionate, zinc butyrate, zinc formate, zinc gluconate, zinc glycolate, zinc lactate, and mixtures thereof. In one embodiment, the zinc salt comprise zinc acetate.

Besides the zinc salt or mixtures thereof, the chemical sterilant can further include an additive or mixtures thereof. The additive can be a pharmaceutically acceptable preservative; buffer; dye; colorant; numbing agent; solvent; detergent; antibiotic; antioxidant; viscolyzing agent; polycation such as polymyxins and their derivatives; protamine; polymer of basic amino acids; insect cecropin; reptilian magainin; cationic leukocyte peptide such as defensins, bactenecins, and bactericidal/permeability-increasing protein; aminoglycoside; chelator such as EDTA, nitrilotriacetic acid, and sodium hexametaphosphate; or any other pharmaceutically acceptable additive that is known in the art.

In another embodiment, the additive can be an amino acid such as alanine, valine, isoleucine, proline, glycine, serine, threonine, asparagine, glutamine, lysine, arginine, histidine, and mixtures thereof. In yet another embodiment, the additive is histidine. In still another embodiment, the additive is dimethyl sulfoxide. In this regard, the chemical sterilant can be a mixture of zinc acetate, histidine, and dimethyl sulfoxide, with a pH in the range of about 6 to 8.

The zinc salt or mixtures thereof is present in the chemical sterilant at a concentration in the range of about 70 to 100 mg/ml. The histidine is present at a concentration in the range of about 0.1 to about 0.3 M. The dimethyl sulfoxide is present at a range of about 0.1-70% by volume.

### EXAMPLE

In this study, 32 beef bull calves (base weight, BW=252±58 lb) and their dams were separated from a larger group of cow-calf pairs for evaluation of an injectable castration method in nursing bull calves. The bull calves average birthdate was Mar. 4, 2014 (range of February 4 to April 4). On Jun. 3, 2014, at about three months of age, the calves were allocated to treatments by bodyweight and birthdate. Twenty-seven bull calves were allocated to 3 injectable castration treatments (n=9 calves/injectable castration treatment) with technicians on-site being blinded to treatments. Treatments were arranged to reflect 3 levels of dosage amounts of chemical sterilant solution that is an aqueous solution of zinc acetate, histidine, and water with a pH about 7. These treatments were identified as Inj1 (73.3 mg/ml zinc acetate, 0.1 mole histidine), Inj2 (86.1 mg/ml zinc acetate, 0.1 mole histidine), and Inj3 (73.3 mg/ml zinc acetate, 0.1 mole histidine). Two bull calves were castrated using knife techniques and 2 bull calves were left intact until the termination of the study at weaning.

Bulls were gathered from pastures, separated from dams, and weighed with no further shrink prior to processing and on 28-day intervals and weaned from dams on Sep. 30, 2014. At each subsequent 28-day interim weight collection and at final castration on Oct. 3, 2014, a blood sample was obtained from the jugular vein into a plain glass vacuum collection tube. Blood was centrifuged and serum stored frozen until analyzed for testosterone concentrations by a commercially available ¹²⁵I radioimmunoassay kit (ImmuChem™ Double Antibody Testosterone, MP Biomedicals, LLC, Solon, Ohio). On days 28, 56, 83, and 122 while the calves were restrained in a hydraulic chute, the thicknesses of the right testicle and scrotum were measured using a digital caliper.

Bodyweight and performance were analyzed using the general linear measures procedure of SAS (SAS Inst. Inc., Cary, N.C.). Serum testosterone concentrations and thicknesses of the right testicle and scrotum were analyzed using repeated measures analyses. An individual animal was the experimental unit. Orthogonal contrasts were used to compare intact vs. castrated, injection vs. knife, and linear and quadratic effects for chemical sterilant dosage. These linear and quadratic effects assume that the solution dosages were arranged in a linear manner for Inj1, Inj2, and Inj3. The researchers continue to be blinded to those details about the injectable treatments.

Performance of bulls and steers between application of treatments on June 3 and weaning on September 30 are presented in Table 1. There were no effects (P≧0.64) of castration or castration method on bodyweight or preweaning average daily gain. Over the course of the experiment, mean average daily gain was nearly or slightly above 2 lb/day for the initial 2 periods (1.95±0.15 and 2.01±0.18 lb/day, for periods 1 and 2, respectively), yet declined to1.63±0.17 lb/day in period 3 and to 0.33±0.15 lb/day in the final period before weaning. The decline in performance during the late summer is due to seasonal deterioration in forage quality and are not related to treatments imposed. Bodyweight at weaning averaged 445±30 lb.

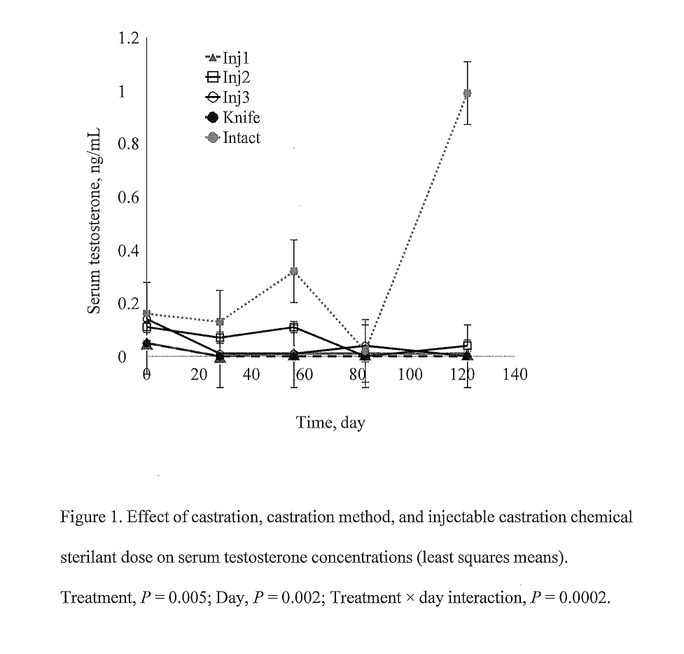

Serum testosterone concentrations are presented in Table 2 and FIG. 1. There was a main effect of treatment (P=0.005) on serum testosterone concentrations. Intact bulls had greater (P<0.001, orthogonal contrast of intact vs. castrated) serum testosterone concentrations than bulls castrated with any method, and there were no differences (P=0.66, orthogonal contrast of injection vs. knife castration) due to castration method. There was a treatment×day interaction (P=0.0002), whereas on day 0 all treatments had similar serum testosterone concentrations, and on day 122 intact bulls had dramatically greater serum testosterone concentrations other castrates, regardless of the method used for castration. There were no differences (P≧0.32, linear and quadratic contrasts for chemical sterilant dosage) in serum testosterone concentrations due to the dosage amounts of the chemical sterilant solution.

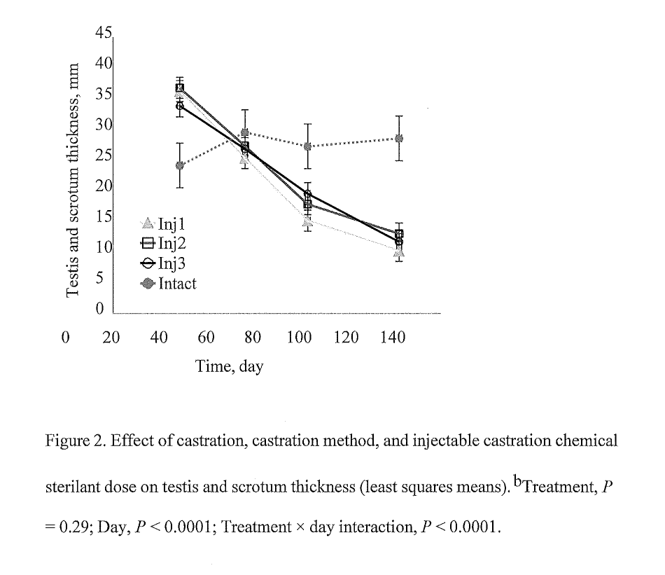

Thicknesses of testis and scrotum are presented in Table 2 and FIG. 2. There was no main effect of treatment (P=0.29). There was a treatment×day interaction (P=0.0001), no change in the thicknesses of the scrotum and testis of intact bulls was observed from day 28 to 122; however, the thicknesses of scrotums and testes for calves given all chemical sterilant dosages decreased as the study progressed. There were no differences (P≧0.39, linear and quadratic contrasts for chemical sterilant dosage) in thicknesses of scrotums and testes due to the dosage amounts of chemical sterilant solution.

There were no differences in growth performance between calves that remained bulls and calves castrated with any of these methods. Serum testosterone and scrotal and testes thickness were greater in intact bulls than in castrates by weaning. There were no differences in these measurements due to the dosage of chemical sterilant used, and the injectable castration method resulted in similar serum testosterone concentrations to calves that had been surgically castrated.

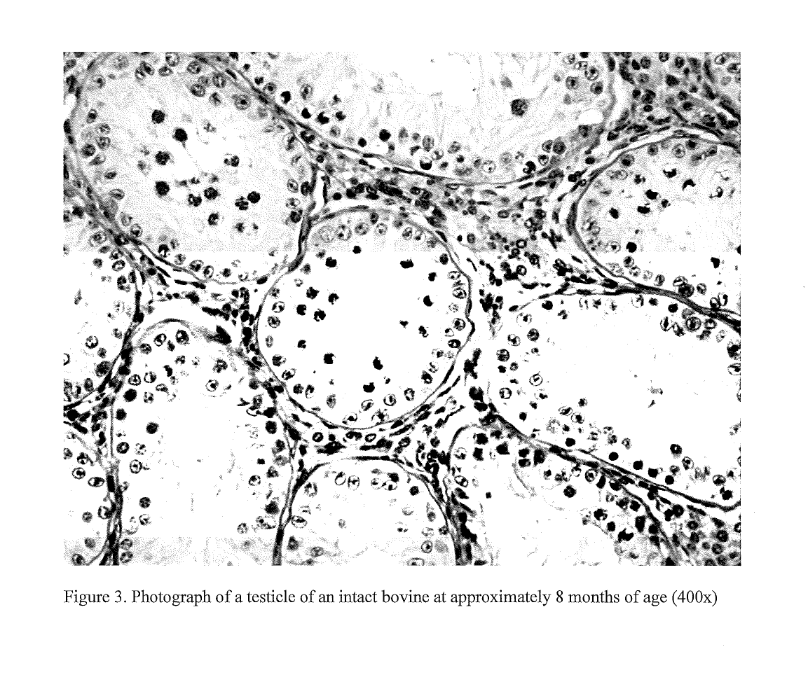

The histopathology of the testicles were also examined FIG. 3 shows a photograph of pubertal testis of an intact bovine (400×) at approximately 8 months of age. The photo shows well-defined seminiferous tubules containing Sertoli cells, spermatogonia, and spermatocytes. No spermatozoa are present. There is normal intertubular space with connective tissue, leydig cells, and lymph sinusoids.

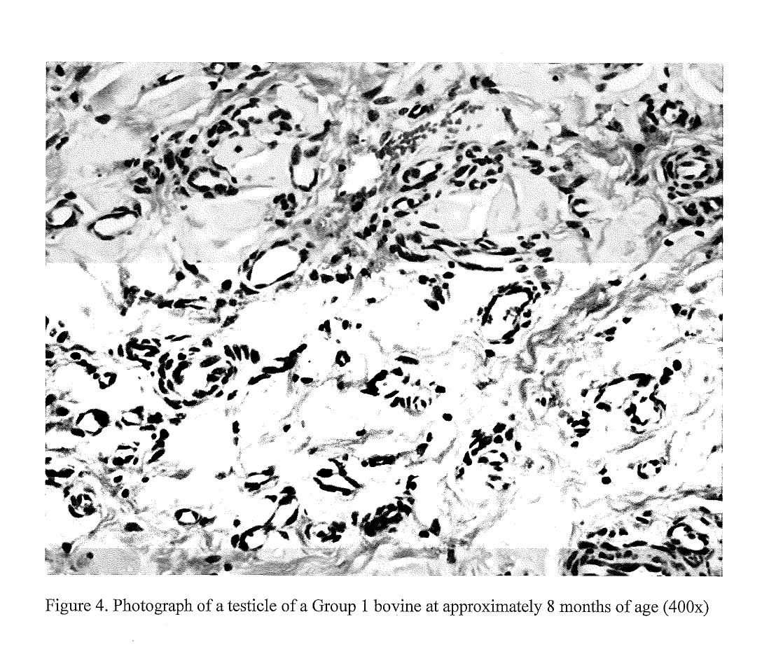

In contrast, an eight month old bovine treated with the present chemical sterilant, inj1, (Group 1) shows evidence of sterility in the photograph of FIG. 4. The testis showed nearly total atrophy with fibrosis and a variety of pathogenic stages including necrosis, total loss of seminiferous epithelium, and loss of Sertoli cells and any germ cells. Also, the testis presented seminiferous tubules that were hypospermatogenic epithelial voculation. Foal lymphocyte infiltration or an inflammatory artery was present in the area.

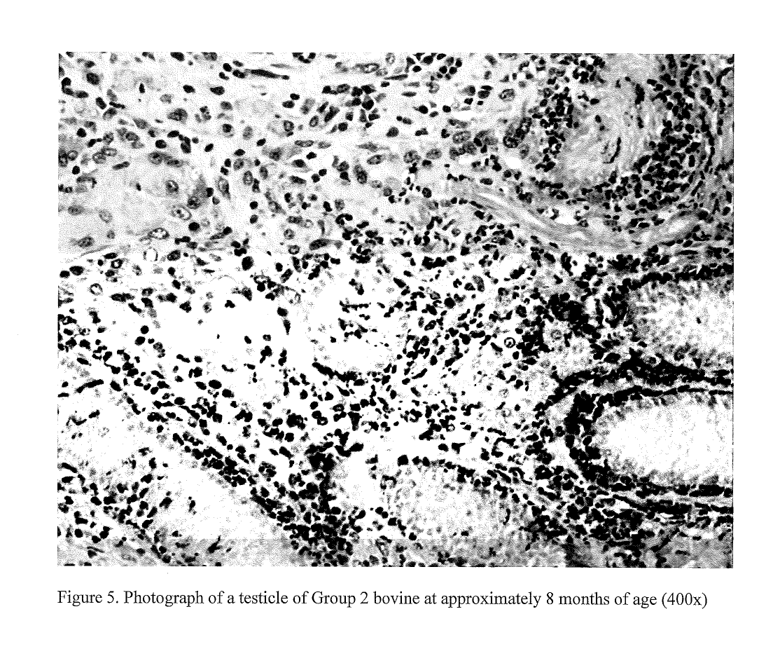

Similarly, an eight month old bovine treated with the present chemical sterilant, inj2, (Group 2) shows evidence of sterility in the photograph of FIG. 5. The testis showed nearly total atrophy with fibrosis and a variety of pathogenic stages including necrosis, total loss of seminiferous epithelium, and loss of Sertoli cells and any germ cells. Also, the testis presented seminiferous tubules that were hypospermatogenic epithelial voculation. Foal lymphocyte infiltration or an inflammatory artery was present in the area.

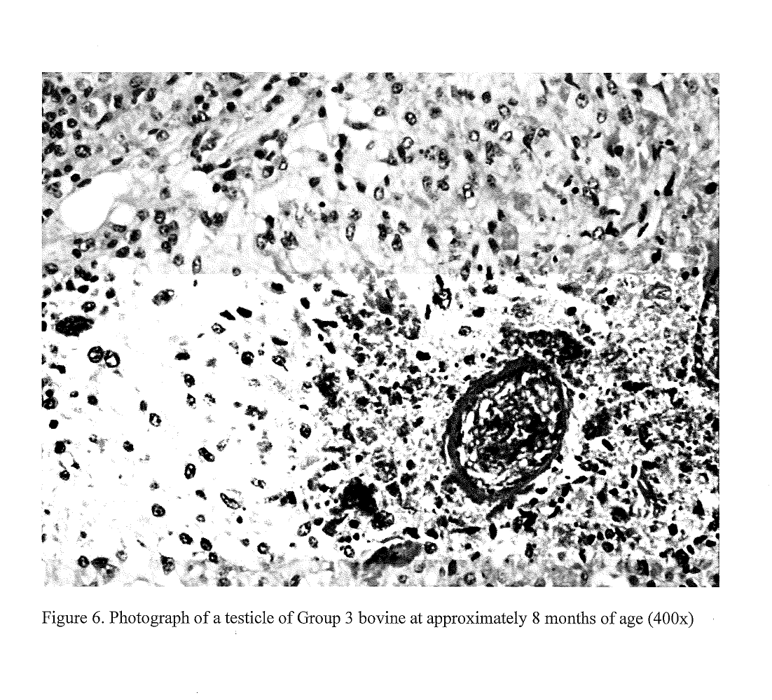

Likewise, an eight month old bovine treated with the present chemical sterilant, inj3, (Group 3) shows evidence of sterility in the photograph of FIG. 6. The testis showed nearly total atrophy with fibrosis and a variety of pathogenic stages including necrosis, total loss of seminiferous epithelium, and loss of Sertoli cells and any germ cells. Also, the testis presented seminiferous tubules that were hypospermatogenic epithelial voculation. Foal lymphocyte infiltration or an inflammatory artery was present in the area.

TABLE 1
Effect of castration, castration method, and injectable castration chemical
sterilant dose on bodyweight and average daily gain of nursing male calves

| | Castration method | | | | | | |
|---|---|---|---|---|---|---|---|
| Item | Inj1 | Inj2 | Inj3 | Knife | Intact | SE | P-value |
| Body weight, lb | | | | | | | |
| June 3 | 286 | 288 | 286 | 251 | 275 | 21.1 | 0.96 |
| July 1 | 338 | 346 | 339 | 308 | 328 | 22.9 | 0.97 |
| July 29 | 398 | 406 | 389 | 366 | 383 | 25.4 | 0.96 |
| August 25 | 440 | 441 | 432 | 410 | 426 | 27.4 | 0.99 |
| September 30 | 451 | 472 | 440 | 432 | 433 | 30.9 | 0.94 |
| Average daily gain, lb | | | | | | | |
| Period 1 | 1.86 | 2.07 | 1.90 | 2.04 | 1.88 | 0.15 | 0.85 |
| Period 2 | 2.12 | 2.13 | 1.78 | 2.07 | 1.98 | 0.18 | 0.64 |
| Period 3 | 1.56 | 1.81 | 1.61 | 1.63 | 1.56 | 0.17 | 0.88 |
| Period 4 | 0.32 | 0.49 | 0.23 | 0.62 | 0.21 | 0.18 | 0.77 |
| Overall | 1.51 | 1.68 | 1.42 | 1.67 | 1.44 | 0.15 | 0.76 |

TABLE 2
Effect of castration, castration method, and injectable castration chemical sterilant
dose on serum testosterone concentrations and testis and scrotum thickness
(least squares means)

| | | | | | | SE | |
|---|---|---|---|---|---|---|---|
| | | | | | | Injection | Intact and |
| | Castration method | | | | | treatments | Knife |
| Item | Inj1 | Inj2 | Inj3 | Knife | Intact | (n = 9/method) | (n = 2/method) |
| Serum testosterone^{a}, ng/mL | | | | | | 0.056 | 0.118 |
| June 3, day 0 | 0.05^{c} | 0.11^{bc} | 0.14^{bc} | 0.05^{c} | 0.16^{bc} | | |
| July 1, day 28 | 0^{c} | 0.07^{bc} | 0.01^{c} | 0^{c} | 0.13^{bc} | | |
| July 28, day 56 | 0.01^{c} | 0.11^{bc} | 0.01^{c} | 0^{c} | 0.32^{b} | | |
| August 25, day 83 | 0.01^{c} | 0^{c} | 0.04^{c} | 0^{c} | 0.02^{c} | | |
| October 3, day 122 | 0.01^{c} | 0.04^{c} | 0^{c} | 0^{c} | 0.99^{a} | | |
| Testis and scrotum thickness^{b}, mm | | | | | | 1.73 | 3.64 |
| July 1, day 28 | 36.2^{ab} | 36.74^{a} | 33.8^{ab} | — | 24.1^{cd} | | |
| July 28, day 56 | 25.3^{c} | 27.3^{c} | 26.8^{c} | | 29.5^{ab} | | |
| August 25, day 83 | 15.1^{ef} | 17.8^{de} | 19.5^{de} | | 27.2^{c} | | |
| October 3, day 122 | 10.2^{g} | 13.01^{g} | 11.7^{fg} | — | 28.5^{bc} | | |

^{a}Treatment, P = 0.005; Day, P = 0.002; Treatment × day interaction, P = 0.0002
^{b}Treatment P = 0.29; Day, P < 0.0001; Treatment × day interaction, P < 0.0001
^{abcdefg}Means without a common superscript differ P < 0.05

What is claimed:

1. A method of performing a non-surgical sterilization of a male animal comprising injecting a chemical sterilant into a testicle of the animal wherein the chemical sterilant comprises an aqueous solution of a pharmaceutically acceptable zinc salt or mixtures thereof having a pH in the range of about 6 to 8.

2. The method of claim 1 wherein the zinc salt comprises a salt selected from the group consisting of zinc acetate, zinc propionate, zinc butyrate, zinc formate, zinc gluconate, zinc glycolate, zinc lactate, and mixtures thereof.

3. The method of claim 1 wherein the zinc salt comprise zinc acetate.

4. The method of claim 1 wherein the chemical sterilant further comprises an additive or mixtures thereof.

5. The method of claim 4 wherein the additive comprises an amino acid selected from the group consisting of alanine, valine, isoleucine, proline, glycine, serine, threonine, asparagine, glutamine, lysine, arginine, histidine, and mixtures thereof.

6. The method of claim 4 wherein the additive comprises histidine.

7. The method of claim 4 wherein the additive further comprises dimethyl sulfoxide.

8. The method of claim 4 wherein the chemical sterilant comprises zinc acetate, histidine, and dimethyl sulfoxide, with a pH in the range of about 6 to 8.

9. The method of claim 1 wherein the zinc salt or mixtures thereof comprises a concentration in the range of about 70 to 100 mg/ml.

10. The method of claim 1 wherein the histidine comprises a concentration in the range of about 0.1 to about 0.3 M.

11. The method of claim 1 wherein the dimethyl sulfoxide comprises a range of about 0.1 to 70% by volume.

12. The method of claim 1 wherein the animal is a bull calf between 3 to 24 months of age.

13. A chemical sterilant comprising dimethyl sulfoxide and an aqueous solution of a pharmaceutically acceptable zinc salt or mixtures thereof having a pH in the range of about 6 to 8.

14. The chemical sterilant of claim 13 wherein the zinc salt comprises a salt selected from the group consisting of zinc acetate, zinc propionate, zinc butyrate, zinc formate, zinc gluconate, zinc glycolate, zinc lactate, and mixtures thereof.

15. The chemical sterilant of claim 13 wherein the zinc salt comprises zinc acetate.

16. The chemical sterilant of claim 13 wherein the chemical sterilant further comprises an additive.

17. The chemical sterilant of claim 13 wherein the additive comprises an amino acid selected from the group consisting of alanine, valine, isoleucine, proline, glycine, serine, threonine, asparagine, glutamine, lysine, arginine, histidine, and mixtures thereof.

18. The chemical sterilant of claim 13 wherein the additive comprises histidine.

19. (canceled)

20. The A chemical sterilant comprising zinc acetate, histidine, dimethyl sulfoxide, and water with a pH in the range of about 6 to 8.
